(19) United States
(12) Patent Application Publication
(10) Pub. No.: US 2006/0074079 A1
(43) Pub. Date: Apr. 6, 2006

(54) Solid pharmaceutical formulations comprising Diacereine and Meloxicam

(75) Inventors: Maria Elena Garcia Armenta, La Estancia (MX); Josefina Santos Murillo, Arco de Zapopan (MX); Victor Guillermo Alvarez Ochoa, La Herradura (MX); Consuelo Flores Mendoza, Sector Hidalgo (MX)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Leopoldo Espinosa Abdala, Zapopan (MX)

(21) Appl. No.: 11/186,031

(22) Filed: Sep. 30, 2005

(30) Foreign Application Priority Data
Oct. 4, 2004 (MX) PA/A/2004/009698

(51) Int. Cl. A61K 31/54; A61K 31/192

(52) U.S. Cl. 514/226.5; 514/569

(57) ABSTRACT

This invention relates to formulations in solid pharmaceutical forms containing diacereine and meloxicam. The present invention provides novel formulations comprising: (a) Diacereine, (b) Meloxicam, (c) one or more anti-adherent agents, (d) one or more disintegrating agents, (e) one or more binder agents, (f) one or more lubricants, (g) one or more diluents, (h) one or more solvents, and (i) any other additive which assists in formulation. The present invention also provides a method for treatment of osteoarthritis, rheumatoid arthritis, gout arthritis, multiple sclerosis, amyotrophic lateral sclerosis and related diseases, in addition of inflammatory processes originated from various etiologies, by administering suitable doses.

### FIELD OF THE INVENTION

Solid pharmaceutical forms are preparations generally disk-shaped, scored and non-scored and in different sizes containing the active principle(s) and additives, which are obtained by compressing powders or granules into tablets, capsules, patches, pessaries, beads, suppositories, troches or lozenges.

### BACKGROUND OF THE INVENTION

ARTHROSIS, also called OSTEOARTHRITIS or JOINT DEGENERATIVE DISEASE, more than an illness, it is a syndrome, the final expression of disturbances produced on the articulation by a diverse group of etiopathogenic processes (excessive or repeating mechanical overload, genetic factors, metabolic or inflammatory processes).

As for economical and social costs, the disease has an enormous importance in the occidental world: it is the most important cause of functional disability regarding processes related to the locomotive apparatus, and the second cause of permanent disability after cardiovascular diseases.

Arthrosis is the most frequent of all joint diseases and its prevalence increases with age. It is estimated that radiological signs of arthrosis are unusual before 40 years of life (2%), they show in 30% of people with an age between 45 and 65 years old and in 68% of people above 65 years old. Typical arthrosis joint disturbances begin in the second decade of life, affecting about 90% of people above 40 years old.

The illness is distributed universally, although there are geographical differences, due in part to genetic factors, environmental correlation and to the different usage of the joints.

Osteoarthritis is the eighth cause of disability worldwide, according to studies realized by WHO.

As for rheumatoid arthritis, this is a disorder with a relatively common prevalence and worldwide distribution affecting adult men and women. It has the potential to severely affect survival, functionality and quality of life of the individual affected with, as well as the ability to keep a satisfactory employment. Mortality due to direct causes or by complications derived from rheumatoid arthritis is still almost twice than observed in the control population, and this trend has kept unchanged for the last four decades. In the Latin-American countries the economical impact derived from this pathology has been underestimated. In the U.S.A. it has been considered as one of the principal reasons for invalidity pension and direct or indirect economical losses.

### SUMMARY OF THE INVENTION

The present invention therefore provides novel formulations comprising: (a) Diacereine, (b) Meloxicam, (c) one or more anti-adherent agents, (d) one or more disintegrating agents, (e) one or more binder agents, (f) one or more lubricants, (g) one or more diluents, (h) one or more solvents, and (i) any other additive which assists in formulation.

Diacereine is 1,8-diacetoxy-3-carboxy-anthraquinone and it can be represented by the formula (I).

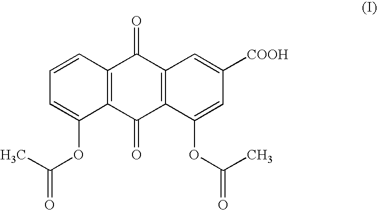

The compound of formula (I) is known by its pharmacological activity as an anti-arthritic, analgesic and anti-inflammatory agent, which acts by inhibiting Interleukine I which has a predominant role in osteoarthritis. Reine, which is diacereine's active metabolite, is an inhibitor of NADH-associated oxidation, namely it interferes with NADH-dehydrogenase complex function and so with mitocondrial oxidation; it also forms chelates with calcium and copper. Currently is indicated for treatment of osteoarthritis, it is a modifier of cartilage structure and it alleviates pain.

The use of anthraquinone derivatives, as in the case of diacereine, in aqueous solution is not convenient since they are not stable enough to water.

Meloxicam is 4-hydroxy-2-methyl-N-(5-methyl-2-thiazolyl)-2H-1,2-benzothiazine-3-carboxamide-1,1-dioxide and can be represented by formula (II):

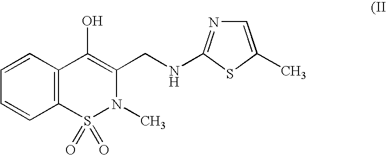

The compound of formula (II) is known by its anti-inflammatory, analgesic and antipyretic activity, and it acts inhibiting the synthesis of prostaglandin with greater potency at the site of inflammation than on kidney and gastric mucosa. Currently is indicated in treatment of rheumatoid arthritis, osteoarthritis, periarthritis in humeral articulation and hip joint, muscular strains, and pain and inflammation in soft tissues and airways with inflammatory processes.

The active ingredient in the formulation is present in an amount in the range from 0.0001% to 95.0% w/w, more preferably from 0.5 to 70.0% w/w for diacereine, and in an amount in the range from 0.0001% to 90.0% w/w, more preferably from 1.0 to 30.0% w/w for Meloxicam.

The present invention is characterized in that the active ingredients combined therein can be present as the anhydrous base or hydrated form or as a physiologically acceptable salt.

### BRIEF DESCRIPTION OF THE DRAWINGS

FIG. 1, shows a graph defining the analgesic effects of diacereine/meloxicam combination vs. diacereine and meloxicam individually.

FIG. 2, shows a graph of Functionality Index Percentage (FI %) in which the analgesic or antinociceptive effect of diacereine/meloxicam formulation vs. diacereine and meloxicam individually, is described.

FIG. 3, shows a graph of analgesic effect 4 hours post-administration of diacereine/meloxicam formulation vs. diacereine and meloxicam individually.

### DETAILED DESCRIPTION OF THE INVENTION

The pharmaceutical formulation can comprise a series of additives or excipients such as, for example, anti-adherents such as colloidal silicon dioxide, calcium sulfate, calcium chloride, talc, corn starch, among others, being the most preferred colloidal silicon dioxide. The anti-adherent can be present in an amount in the range from 0.0001% to 10.0%. Disintegrating agents such as corn starch, alginic acid, cellulose and its derivatives, povidone, sodium crosscarmelose, sodium starch glycolate, among others, the most preferred being sodium crosscarmelose. The disintegrating agent can be present in an amount ranging from 0.0001% to 30.0%. Lubricant agents such as stearic acid, magnesium stearate, talc, among others, the most preferred being talc. The lubricant agent can be present in an amount ranging from 0.0001% to 10.0%. Diluent agents such as lactose, mannitol, calcium phosphate, microcrystalline cellulose, calcium sulfate, sucrose for compression, corn starch, among others, the most preferred being sucrose for compression. The diluent agent can be present in an amount ranging from 1% to 99%. Coatings such as methacrylates, polyvinyl alcohol, derivatives of cellulose, blends of polymers and polysaccharides for coating by film formation utilizing aqueous or organic solvents, with coloring agents, flavoring agents, sugars and any other ingredient used in applications of film forming, coating and making, such as talc, titanium dioxide, among others. Capsules can be hard or soft gelatin capsules, preferably hard gelatin capsules. Polar and non-polar solvents such as water, ethyl alcohol, isopropyl myristate, polyoxypropylenes, propyleneglycol, polyethyleneglycol, glycerol, 70% sorbitol, polyethyleneglycols, mineral oil, petrolatum, lanoline, vegetable waxes, animal waxes, vegetable oils such as olive oil, cottonseed oil, corn oil, among others, preferably ethyl alcohol and polar solvents such as water are used. Final formulation can contain from 1% to 60% w/v of solvent. Binder agents such as polyvidone, tragacanth, acacia, starch, methylcellulose, among others, preferably polyvidone. The binder can be present in an amount ranging from 0.0001% to 20.0%.

Formulations can be contained in blisterpacks made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil; they can also be contained in blisterpack made of 2 sheet of aluminum foil bonded together to form the blisterpack, or they can be in cellopolyal film bonded together with aluminum foil. In vials of suitable capacity varying from 5 ml to 500 ml, elaborated from high and/or low density polyethylene, polyethylene terephthalate, polyvinyl chloride, polypropylene, polystyrene, Type I, II, III and IV glass, among others, with or without color. The cap may be of the following types: tamperproof, threaded, cap-to-cap, child proof, elaborated from high and/or low density polyethylene, polyethylene terephthalate, polyvinyl chloride, polypropylene, polystyrene, with or without color. Blisterpack made of polyvinyl chloride coated with polyvinylidene bound to aluminum is preferred.

The elaboration of the different pharmaceutical forms is carried out mixing the active ingredients with the corresponding additives in the adequate concentrations.

Upon administration of diacereine, it undergoes a complete deacetylation process, and it becomes in its active metabolite Reine before it passes to systemic circulation. According to pharmacokinetics studies it binds to proteins in 99%. About 50 to 60% of diacereine orally administered was excreted through kidney as reine and its conjugates

Administering 15 mg of meloxicam every 24 hours will allow to reach peak concentrations of 1.6 μg/ml in plasma. Concentrations at equilibrium can be reached in 3 to 5 days. 99% of meloxicam binds to plasma proteins. The drug has a good distribution in the body, but particularly it achieves high penetration in synovial fluid, reaching levels that are equivalent to the half of concentrations in plasma. Meloxicam is metabolized mainly through oxidation of the methyl group in the thiazolyl molecule. About 50% of the dose is eliminated through urine and the remaining is excreted in feces. The clearance half-life is 20 hours.

It is recommended for treatment of osteoarthritis and related disorders, to initiate with 50 mg of diacereine daily during the first for weeks of treatment, which can prolong for al least 6 months and clinical trials had shown that the drug can be administered to 2 years without having safety problems. For how long should the treatment be continued is a decision taken by the attendant physician. For meloxicam a dose of 15 mg every 24 hours is recommended in adults.

Therefore, the present invention provides a pharmaceutical formulation in solid form, which provides from 5 mg to 150 mg of diacereine and from 1 to 30 mg of meloxicam in suitable doses.

### EXAMPLES

The following non-limiting examples will assist to illustrate the present invention:

### Example 1

| | | |
|---|---|---|
| 1) Diacereine | 23.00% | w/w |
| 2) Meloxicam | 7.00% | w/w |
| 3) Sodium crosscarmelose | 1.00% | w/w |
| 4) Polyvidone k-30 | 1.00% | w/w |
| 5) Colloidal silicon dioxide | 0.50% | w/w |
| 6) Talc | 3.00% | w/w |
| 7) Sucrose for compression | 64.50% | w/w |

Tablets are elaborated as follows: meloxicam is mixed with colloidal silicon dioxide, then diacereine and sodium crosscarmelose are added and mixed. Next, polyvidone k-30 is added and mixed. Then sucrose for compression is added and mixed. Finally, the talc is added and mixed. Proceed to tableting.

### Example 2

| | | |
|---|---|---|
| 1) Diacereine | 50.00% | w/w |
| 2) Meloxicam | 15.00% | w/w |
| 3) Sucrose for compression | 24.30% | w/w |
| 4) Polyvidone k-30 | 3.00% | w/w |
| 5) Colloidal silicon dioxide | 0.50% | w/w |
| 6) Sodium crosscarmelose | 5.00% | w/w |
| 7) Talc | 2.20% | w/w |

Powder elaboration is carried out as follows: Diacereine is mixed with colloidal silicon dioxide, to this meloxicam and sodium crosscarmelose are added and mixed. Then polyvidone k-30 and sucrose for compression are added and mixed. Finally, talc is added and mixed. Proceed to encapsulation.

Next, the clinical study of meloxicam/diacereine combination is provided, which is carried out by oral route.

Determination of Potency, Effectiveness and Type of Analgesic Synergism Produced by Blend of Meloxicam+Diacereine Orally Administered, on Arthritis

Pre-Clinical Study of Diacereine/Meloxicam Blend

Antecedents

A great number of non-steroidal anti-inflammatory analgesics (NSAIA) are generally used for treatment of osteoarthritis as a first line therapy, however, their clinical activity is limited by several side effects. Osteoarthritis is a degenerative process of joints, characterized by cartilage progressive destruction and erosion, and it is associated with age. The articular cartilage degenerates and eventually is worn out the bone surface, this and subsequent changes produce pain and limitations on the movement of affected patients. NSAIA's provide general symptomatic alleviation in patients affected with osteoarthritis, but those agents decrease prostaglandin synthesis. Unlike NSAIA's, diacereine can stimulate or not affect prostaglandin synthesis and it acts by inhibiting Interleukin-1, therefore it has a potential use as an alternative.

It is believed that inflammatory mediators such as Interleukin-1 (IL-1) play an important role in joint diseases such as osteoarthritis and rheumatoid arthritis. Diacereine, an anthraquinone derivative, is a compound with an unusual mechanism of action, it is an inhibitor of IL-1 that exhibits analgesic, antipyretic and anti-inflammatory activity, affecting pain threshold in rats affected with limb edema and on hyperpyrexia on rabbits (Kay et al., 1980). Diacereine and its metabolite (reine) inhibit Interleukin-1 beta production in human monocytes in vitro (Berdah et al., 1993). Although it inhibits IL-1 that mediates colagenase production in articular cartilage (Boittin et al., 1993), it does not disrupt the activity of cyclooxygenase (COX) from kidney or platelet (LaVilla et al., 1989).

The association of diacereine/meloxicam is an association that has the potential of therapeutic use in patients affected with disorders such as osteoarthritis and rheumatoid arthritis. It is expected, by virtue of the type of analgesic that meloxicam is (mainly peripheral action), a suitable analgesic synergism when it is associated with diacereine (an active agent with mode of action different to prostaglandin inhibition).

Procedure

The experiments were carried out using male Wistar rats having a body weight between 180 and 200 g. All of the experimental procedures were carried out following recommendations from The Committee for Research and Ethical Issues of the International Association for the Study of Pain (Covino et al., 1980) and Guidelines on Ethical Standards for Investigations of Experimental Pain in animals (Zimmermann 1983). The number of test animals was taken to the minimal (6 animals for each experimental aspect). Animals were kept in a room with light/darkness cycles. Rats were fastened twelve hours prior the beginning of experiments, leaving only free access to water. All the experiments were run during the light phase, using the animals only once.

Evaluation of Analgesic Activity

The evaluation of analgesic effects was carried out using the Pain Induced Functional Impairment model in the Rat (PIFIR) model. Animals were anesthetized in a glass desiccator saturated with ether vapors. Gout arthritis was induced, upon application of a intra-articular injection of 0.05 ml of uric acid suspended in mineral oil, in the right-side hind leg, exactly in the femoral-tibio-patellar joint. Immediately after, an electrode was attached to each hind leg in the middle of each pad. Rats were allowed to recover from anesthesia and afterwards were placed in a rotating stainless steel cylinder of 30 cm in diameter. The cylinder was rotated at 4 rpm, forcing the rats to walk for 2 minutes every 15 minutes during the following hour, and every half an hour, for 3 more hours, for a total of 4 hours after onset of total dysfunction. Variable measured was contact time of each hind leg of every rat in the cylinder.

Experimental Protocol

Analgesic effects produced by meloxicam, diacereine or meloxicam/diacereina blend were determined and analyzed individually once gout arthritis was established in rats. Rats were administered with each of the compounds orally. From the moment when the uric acid was administered in the joint to produce a gout type dysfunction and alteration, 2.5 hours were allowed to pass in order to produce total dysfunction (gout arthritis was already complete 2.5 hours after uric acid was administered). This period of time (2.5 hours after administration of uric acid) was considered as time “0”, in order to administer in this point of time the antinociceptive or analgesic treatment and the temporary course of each treatment were determined during the following 4 continuous hours. For each treatment a “n” value of 6 was utilized.

Data Disclosure

Data are expressed as Percentage of Functionality Index (FI %). This FI % is the ratio obtained dividing contact time of the limb with uric acid by the contact time of the contralateral limb of the same rat and this is multiplied by 100. Temporal course(TC) curves are constructed when graphing FI % vs. time (h); the analgesic or antinociceptive effect was estimated as FI % recovery. The accumulated analgesic effect during the total observation period (4 h), was determined as area under curve (AUC) of TC, using trapezoidal rule (Rowland and Toser, 1989), and it also was used to construct dose-response curves (DRC).

### RESULTS AND CONCLUSIONS

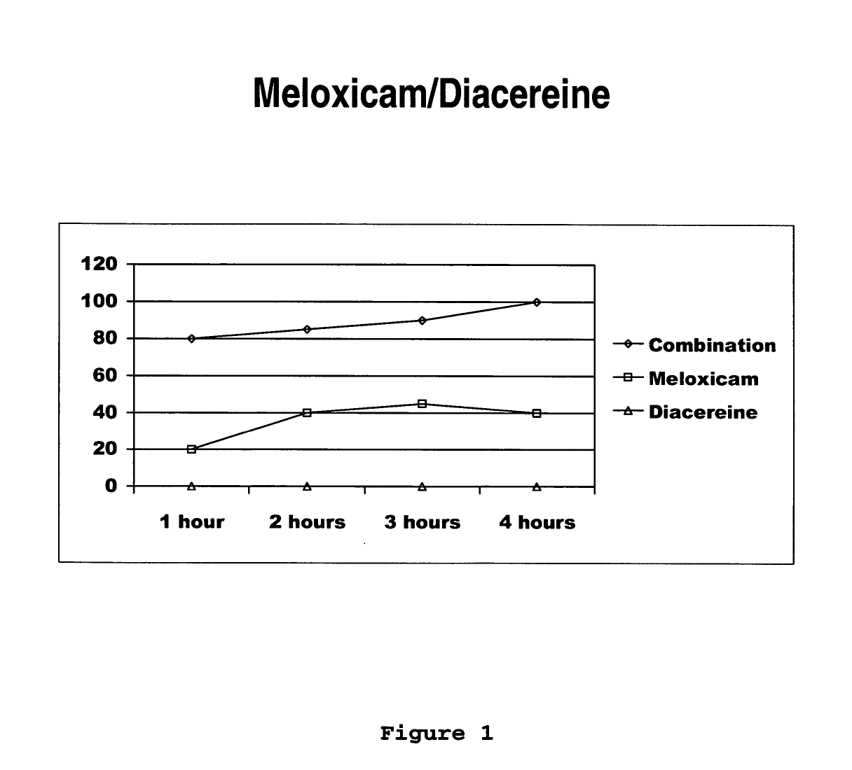

1. As for TC efficacy: Diacereine was unable to generate analgesic effects (FIG. 1), whereas the blend of Diacereine/meloxicam exhibit superior efficacy compared to that showed by meloxicam when administered in single doses.

2. As for the onset of action: Meloxicam generated its effect, in general, in a more delayed manner.

3. Regarding duration of analgesic effect: analgesic effects were analyzed during 4 continuous hours: meloxicam and the blend tend to maintain the Emax obtained up to the 4 continuous hours.

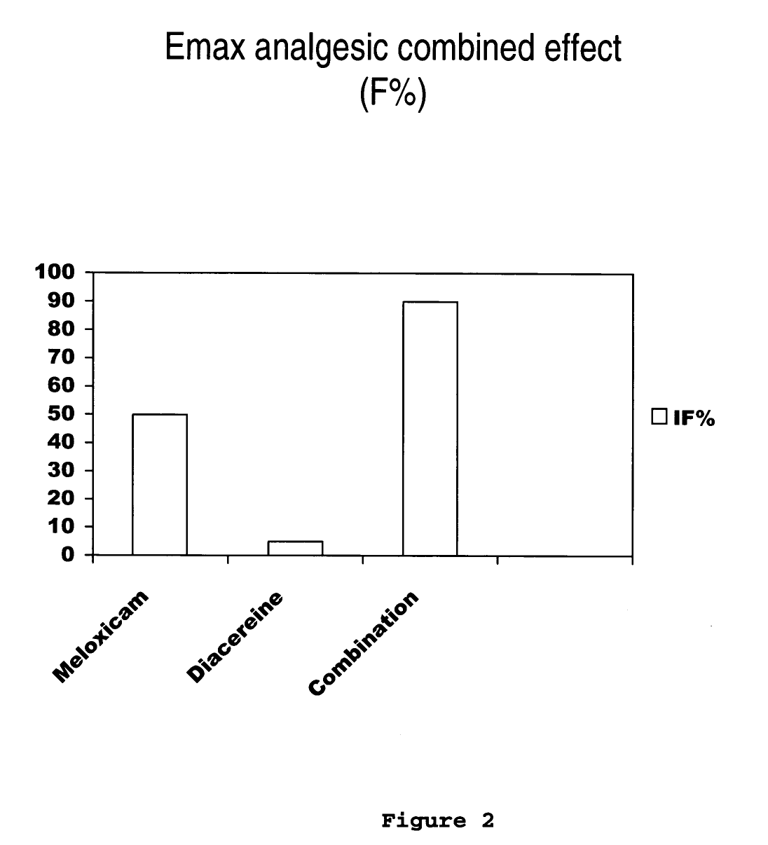

4. As for global analgesic efficacy (GAE) evaluated during 4 continuous hours: The blend exhibits greater efficacy compared to meloxicam (FIG. 2).

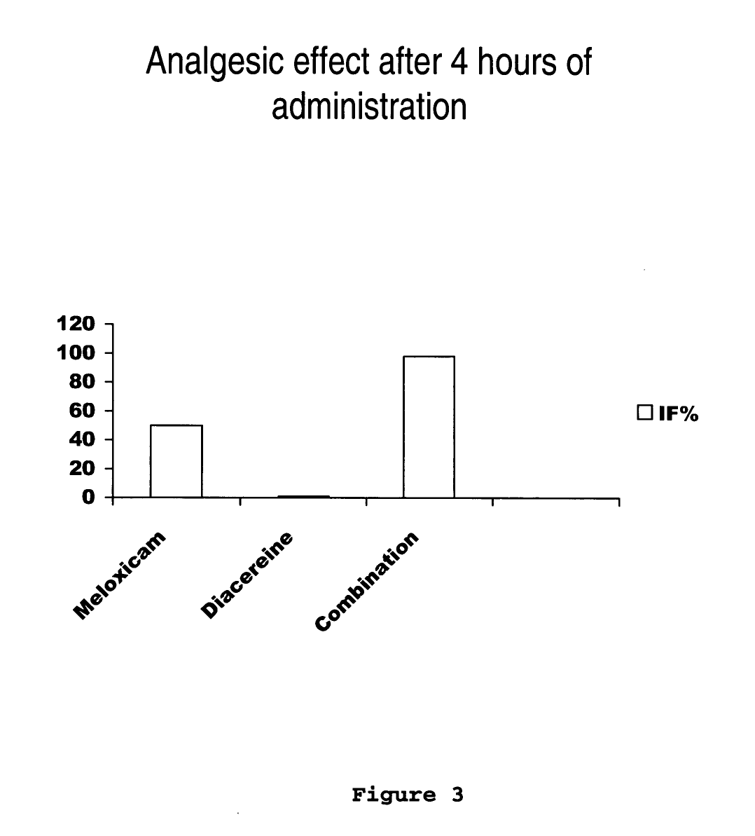

5. As for analgesic potency, meloxicam was more potent than diacereine alone, but DRC's showed that the blend was more potent than meloxicam (FIG. 3).

Under the experimental conditions and pain established in the PIFIR model, the blend of diacereine/meloxicam produced an important improvement in analgesic efficacy an therapeutic coverage compared to the single administration of meloxicam. Combining small doses it is possible to overcome the efficacy of meloxicam.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” 'including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A pharmaceutical formulation comprising: (a) Diacereine, (b) meloxicam, (c) one or more anti-adherent agents, (d) one or more disintegrating agents, (e) one or more binder agents, (f) one or more lubricants, (g) one or more diluents, (h) one or more solvents, and (i) any other additive which assists in formulation.

2. The formulation according to claim 1, wherein the concentration of drug in the formulation is present in an amount in the range from 0.0001% to 95.0% w/w, more preferably from 0.5 to 70.0% w/w for diacereine, and in an amount in the range from 0.0001% to 90.0% w/w, more preferably from 1.0 to 30.0% w/w for Meloxicam.

3. The formulation according to claim 2, wherein the drugs combined therein can be present as the base form or as a physiologically acceptable salt, being the most preferred base diacereine for diacereine and base meloxicam for meloxicam.

4. A formulation according to claim 3, wherein the solid pharmaceutical form may contain a series of additives or excipients selected from the group consisting of anti-adherents such as colloidal silicon dioxide, calcium sulfate, calcium chloride, talc, corn starch, among others, being the most preferred colloidal silicon dioxide, wherein the anti-adherent can be present in an amount in the range from 0.0001% to 10.0%, further comprises disintegrating agents such as corn starch, alginic acid, cellulose and its derivatives, povidone, sodium crosscarmelose, sodium starch glycolate, among others, the most preferred being sodium crosscarmelose, wherein the disintegrating agent can be present in an amount ranging from 0.0001% to 30.0%; further comprises lubricant agents selected from the group consisting of stearic acid, magnesium stearate, talc, among others, the most preferred being talc, and also comprises a lubricant agent that can be present in an amount ranging from 0.0001% to 10.0%, wherein further comprises diluents such as lactose, mannitol, calcium phosphate, microcrystalline cellulose, calcium sulfate, sucrose for compression, corn starch, among others, the most preferred being sucrose for compression, it also comprises a diluent agent that can be present in an amount ranging from 1% to 99%, and it also comprises a coating selected from the group consisting of methacrylates, polyvinyl alcohol, derivatives of cellulose, blends of polymers and polysaccharides for coating by film formation utilizing aqueous or organic solvents, with coloring agents, flavoring agents, sugars and any other ingredient used in applications of film forming, coating and troche making.

5. A pharmaceutical formulation according to claim 4, wherein the pharmaceutical formulation in solid form may also contain others components such as: binders selected from the group consisting of polyvidone, tragacanth, acacia, starch, methylcellulose, among others.

6. A pharmaceutical formulation according to claim 5, wherein the pharmaceutical formulation in solid form may also contain one or more polar and non-polar solvents selected from the group consisting of water, ethyl alcohol, isopropyl myristate, propylene glycol, polyethylene glycol, glycerol, sorbitol solution, polyethylene glycol, among others, wherein the final formulation contains from 1% to 60% w/v of solvent.

7. A tablet comprising the formulation according to claim 1, wherein the pharmaceutical form in a tablet is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

8. A tablet comprising the formulation according to claim 2, wherein the pharmaceutical form in a tablet is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

9. A tablet comprising the formulation according to claim 3, wherein the pharmaceutical form in a tablet is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

10. A tablet comprising the formulation according to claim 4, wherein the pharmaceutical form in a tablet is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

11. A tablet comprising the formulation according to claim 5, wherein the pharmaceutical form in a tablet is contained in blisterpack made of polyvinyl chloride (PVC) film with a thickness from 200μ to 250 μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

12. A tablet comprising the formulation according to claim 6, wherein the pharmaceutical form in a tablet is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

13. A capsule comprising the formulation according to claim 1, wherein the encapsulated pharmaceutical form is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

14. A capsule comprising the formulation according to claim 2, wherein the encapsulated pharmaceutical form is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

15. A capsule comprising the formulation according to claim 3, wherein the encapsulated pharmaceutical form is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

16. A capsule comprising the formulation according to claim 4, wherein the encapsulated pharmaceutical form is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

17. A capsule comprising the formulation according to claim 5, wherein the encapsulated pharmaceutical form is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

18. A capsule comprising the formulation according to claim 6, wherein the encapsulated pharmaceutical form is contained in blisterpack made of polyvinyl chloride (PVC) film with a thickness from 200μ to 250μ, which can or cannot be coated with polyvinylidene chloride (PVDC) with a weight from 25 g/m² to 120 g/m² and bonded with aluminum foil, wherein it may also be contained in blisterpack, made of 2 sheet of aluminum foil bonded together to form the blisterpack, or they can be in cellopolyal film bonded together with aluminum foil.

19. A formulation according to claim 7, wherein it can be contained in vials of suitable capacity varying from 5 ml to 500 ml, elaborated from high and/or low density polyethylene, polyethylene terephthalate, polyvinyl chloride, polypropylene, polystyrene, Type I, II, III and IV glass, among others, with or without color.

20. A formulation according to claim 8, wherein it can be contained in vials of suitable capacity varying from 5 ml to 500 ml, elaborated from high and/or low density polyethylene, polyethylene terephthalate, polyvinyl chloride, polypropylene, polystyrene, Type I, II, III and IV glass, among others, with or without color.

21. A method for the treatment of a disease selected from the group consisting of osteoarthritis, rheumatoid arthritis, gout arthritis, multiple sclerosis, amyotrophic lateral sclerosis and related diseases, and inflammatory processes originated from various etiologies, comprising administering a therapeutically effective amount of the formulation of claim 1.

22. A method for the treatment of a disease selected from the group consisting of osteoarthritis, rheumatoid arthritis, gout arthritis, multiple sclerosis, amyotrophic lateral sclerosis and related diseases, and inflammatory processes originated from various etiologies, comprising administering a therapeutically effective amount of the formulation of claim 2.

23. A method for the treatment of a disease selected from the group consisting of osteoarthritis, rheumatoid arthritis, gout arthritis, multiple sclerosis, amyotrophic lateral sclerosis and related diseases, and inflammatory processes originated from various etiologies, comprising administering a therapeutically effective amount of the formulation of claim 3.

24. A method for the treatment of a disease selected from the group consisting of osteoarthritis, rheumatoid arthritis, gout arthritis, multiple sclerosis, amyotrophic lateral sclerosis and related diseases, and inflammatory processes originated from various etiologies, comprising administering a therapeutically effective amount of the formulation of claim 4.

25. A method for the treatment of a disease selected from the group consisting of osteoarthritis, rheumatoid arthritis, gout arthritis, multiple sclerosis, amyotrophic lateral sclerosis and related diseases, and inflammatory processes originated from various etiologies, comprising administering a therapeutically effective amount of the formulation of claim 5

26. A method for the treatment of a disease selected from the group consisting of osteoarthritis, rheumatoid arthritis, gout arthritis, multiple sclerosis, amyotrophic lateral sclerosis and related diseases, and inflammatory processes originated from various etiologies, comprising administering a therapeutically effective amount of the formulation of claim 6.

27. The pharmaceutical formulation in solid form according to claim 1, wherein it comprises from 5 mg to 150 mg of diacereine and from 1 to 30 mg of meloxicam in suitable doses.
